(19) United States
(12) Patent Application Publication
(10) Pub. No.: US 2025/0121023 A1
(43) Pub. Date: Apr. 17, 2025

(54) COMPOSITION FOR PREVENTING, IMPROVING, OR ALLEVIATING SYMPTOMS RELATING TO QI DEFICIENCY AND/OR BLOOD DEFICIENCY

(71) Applicant: SUMITOMO CHEMICAL COMPANY, LIMITED, Tokyo (JP)

(72) Inventors: Katsunori SASAKI, Tokyo (JP); Keiko HIRANO, Tokyo (JP); Takamitsu YOSHIDA, Nagoya-shi (JP)

(73) Assignee: SUMITOMO CHEMICAL COMPANY, LIMITED, Tokyo (JP)

(21) Appl. No.: 18/715,281

(22) PCT Filed: Nov. 25, 2022
(86) PCT No.: PCT/JP2022/043539

§ 371 Date: Dec. 20, 2024

(30) Foreign Application Priority Data
Dec. 3, 2021 (JP) 2021-196994

(51) Int. Cl. A61K 36/9066; A61K 31/718; A61K 36/23; A61K 36/254; A61K 36/286; A61K 36/287; A61K 36/484; A61K 36/487; A61K 36/534; A61K 36/535; A61K 36/54; A61K 36/67; A61K 36/732; A61K 36/752; A61K 36/82; A61K 36/8945; A61K 36/899; A61K 36/8994; A61K 36/9068; A61P 3/00; A61P 13/00; A61P 25/20

(52) U.S. Cl. CPC A61K 36/9066; A61K 31/718; A61K 36/23; A61K 36/254; A61K 36/286; A61K 36/287; A61K 36/484; A61K 36/487; A61K 36/534; A61K 36/535; A61K 36/54; A61K 36/67; A61K 36/732; A61K 36/752; A61K 36/82; A61K 36/8945; A61K 36/899; A61K 36/8994; A61K 36/9068; A61P 3/00; A61P 13/00; A61P 25/20

(57) ABSTRACT

The composition includes the following active components: component (1): at least one kind of honey-fried licorice root, licorice root, Dioscorea japonica Thunberg, jujube fruit, malt starch syrup, and ginseng root; component (2): at least one of wild turmeric, longan aril, zedoary root, Suberect Spatholobus stem, Luffa vegetable sponge, Sparganium rhizome, safflower, and Artemisia anomala; component (3): at least one of tea leaf, mentha herb, mulberry leaf, chrysanthemum flower, and Koushi; and component (4): at least two of ripe tangerine peel, Psoralea corylifolia fruit, Chaenomeles speciosa fruit, Piper longum, cinnamon twig, Perilla, ginger root, coriander, Allium fistulosum Linne, Zizyphi Spinosi Semen, Chinese arborvitae kernel, plantago seed, plantago herb, coix seed, corn silk, belvedere Fruit, Benincasa seed, knotgrass, snowbellleaf tickclover herb, cinnamon, processed ginger, fennel, Zanthoxylum fruit, Piper nigrum, Eleutherococcus senticosus Rhizome, and Artemisia leaf.

### TECHNICAL FIELD

The present invention relates to a composition for preventing, improving, or alleviating symptoms relating to qi deficiency and/or blood deficiency.

### BACKGROUND ART

In Chinese medicine that is a representative traditional medicine system and in Kampo medicine derived from the Chinese medicine, the theory of “life force (qi), blood, and colorless bodily fluids” is systematized as a basic concept for interpreting the physical condition and constitution and the effects of the foods and pharmaceuticals ingested. This is a way of thinking that lists three elements of life force, blood, and colorless bodily fluids as the elements that make up the body, and classifies the physical condition and constitution as a result of imbalances such as lack of the elements, and stagnation of flow.

Among them, the term “life force” refers to energy of the living body, and the “life force” has a function, is intangible, and is considered to circulate throughout the whole body through channels to nourish and protect all the entrails, and to maintain the life. It can be interpreted in Western medicine that the “life force” is involved in a function of a signal transduction system of the living body, mainly such as a nervous system, or an endocrine system. Abnormalities in the “life force” include “qi deficiency” in which the life force is insufficient due to impaired production or excessive exhaustion of the life force, “upward impulse of life force (qi counterflow)” that indicates a condition in which the life force has ascended from the lower body to the upper body, and “depressed” in which the life force is stagnant in various sites, and such abnormalities are understood in association with various health abnormalities (Non-Patent Documents 1 and 2).

Further, the term “blood” phenomenally refers to blood, and is a concept including a function of blood. The “blood” circulates throughout the whole body due to the functioning of the “life force” to bring necessary nutrients to each tissue, and is considered to be a source that creates a shape of the body. Abnormalities in the “blood” include “blood deficiency” that is a pathological condition in which the amount of blood is insufficient, and “blood stasis” that is a pathological condition in which the circulation of blood is impaired and the blood has stagnated, and such abnormalities are understood in association with various health abnormalities (Non-Patent Documents 1 and 2).

The qi deficiency, that is, a lack of “life force” is considered to be caused based on the decline in gastrointestinal function and digestive and absorption function, and refers to a condition of being weak, lacking in energy, and being unable to be active. Examples of the symptoms of discomfort associated with the qi deficiency include malaise, listlessness, fatigability, chronic fatigue, daytime sleepiness, anorexia, susceptibility to infection (easy to catch a cold), hypoplasia, feeling of cold, cold extremity, weakness, diarrhea, swelling, dyspepsia, thinness, dry skin, excessive sweating, mental instability represented by mood depression, friability, abnormality in urine volume due to kidney qi deficiency, that is, abnormalities in voiding function, frequent urination, enuresis, urinary incontinence, and nocturnal enuresis (Non-Patent Documents 1 to 4).

Further, it is presumed that the blood deficiency, that is, insufficiency of the amount of “blood” is caused by the abnormalities in a blood/immune system, and examples of the symptoms of discomfort associated with the blood deficiency include anemia, reduced immunocompetence, poor concentration, insomnia, sleep disorder, hair coming off easily, visual fatigue, dryness and rough of skin, abnormality of nails, cramp, paresthesia, oligomenorrhea, menstrual irregularity, and ill complexion (Non-Patent Documents 1 and 2).

In Kampo medicine, the principle is dialectic order, that is, to identify the cause of disease and administer the prescription corresponding thereto. For symptoms relating to qi deficiency and/or blood deficiency, various herbal medicines (mixed herbal medicines) have been proposed. For example, such herbal medicines include shikunshi-to for qi deficiency-related symptoms, shimotsu-to for blood deficiency-related symptoms, and the like (Non-Patent Documents 1 and 2). These herbal medicines contain multiple herbal medicine components, and are taken according to a prescription by a doctor.

### PRIOR ART DOCUMENTS

### Non-Patent Document

Non-Patent Document 1: Naotoshi Shibahara, Basic terms in Kampo medicine, Farumashia, Vol. 47, No. 5 (2011), p. 408-412
Non-Patent Document 2: Kunio Matsuda, Kazumoto Inaki (editor), Kampo for clinicians (basic edition) (1987), Current Therapy, p. 12-14
Non-Patent Document 3: Seishiro Sento, Systematic Traditional Chinese Medicine Course Digest Part 4, Physiological function and ataxia of qi, body fluid, and blood, Clinical Journal of Traditional Chinese Medicine, Vol. 23, No. 3 (2002), p. 46-54
Non-Patent Document 4: Terutane Yamada, Takejiro Hashimoto (author), Shintoku Fukushima (editor), Illustrated Oriental Medicine <Decoction Edition I Pharmacopoe> (1984), TOKYO INSHOKAN PRINTING CO., LTD, p. 27-28

### SUMMARY OF THE INVENTION

### Problems to be Solved by the Invention

An object of the present invention is to provide a composition including a herbal medicine component for preventing, improving, or alleviating symptoms relating to qi deficiency and/or blood deficiency.

### Means for Solving the Problems

As a result of intensive studies to achieve the above object, the present inventors have found that it is possible to prevent, improve, or alleviate various symptoms relating to qi deficiency and/or blood deficiency by selecting and mixing groups of herbal medicines with specific medicinal benefit coverages and nature tastes. The present invention has been completed through further investigation based on this finding, and provides a novel composition including a herbal medicine component.

That is, for example, the present invention is as follows.

[1] A composition, including as active components the following components (1), (2), (3), and (4):
component (1): at least one kind of herbal medicine selected from the group consisting of honey-fried licorice root, licorice root, Dioscorea japonica Thunberg, jujube fruit, malt starch syrup, and ginseng root;
component (2): at least one kind of herbal medicine selected from the group consisting of wild turmeric, longan aril, zedoary root, Suberect Spatholobus stem, Luffa vegetable sponge, Sparganium rhizome, safflower, and Artemisia anomala;
component (3): at least one kind of herbal medicine selected from the group consisting of tea leaf, mentha herb, mulberry leaf, chrysanthemum flower, and Koushi; and
component (4): at least two kinds of herbal medicines selected from the group consisting of ripe tangerine peel, Psoralea corylifolia fruit, Chaenomeles speciosa fruit, Piper longum, cinnamon twig, Perilla, ginger root, coriander, Allium fistulosum Linne, Zizyphi Spinosi Semen, Chinese arborvitae kernel, plantago seed, plantago herb, coix seed, corn silk, belvedere Fruit, Benincasa seed, knotgrass, snowbellleaf tickclover herb, cinnamon, processed ginger, fennel, Zanthoxylum fruit, Piper nigrum, Eleutherococcus Senticosus Rhizome, and Artemisia leaf.

[2] The composition described in [1], in which the composition is for use in preventing, improving, or alleviating symptoms relating to qi deficiency and/or blood deficiency.

[3] The composition described in [2], in which the symptoms relating to qi deficiency and/or blood deficiency are at least one selected from the group consisting of chronic fatigue, fatigability, malaise, listlessness, abnormality in urine volume, insomnia, and sleep disorder.

[4] The composition described in any one of [1] to [3], in which the composition is a food composition or a pharmaceutical composition.

[5] A method for preventing, improving, or alleviating symptoms relating to qi deficiency and/or blood deficiency, including administering the composition described in any one of [1] to [4] to a subject in need thereof.

[6] The composition described in any one of [1] to [4], in which the composition is for use in preventing, improving, or alleviating symptoms relating to qi deficiency and/or blood deficiency.

[7] Use of the composition described in any one of [1] to [4], in producing a composition for preventing, improving, or alleviating symptoms relating to qi deficiency and/or blood deficiency.

[8] Use of the composition described in any one of [1] to [4], in preventing, improving, or alleviating symptoms relating to qi deficiency and/or blood deficiency.

### Effect of the Invention

By mixing herbal medicines having specific medicinal benefit coverages and nature tastes, the composition of the present invention generates life force, acts on the middle energizer related to resistance/frailty, nourishes the life force to supplement qi deficiency, and further supplements blood deficiency by a function of the supplemented life force, and at the same time, the composition synergistically exerts an ataractic tranquilizing effect, and can prevent, improve, or alleviate various symptoms relating to the qi deficiency and/or the blood deficiency. The composition of the present invention is particularly effective for symptoms such as chronic fatigue, fatigability, malaise, listlessness, abnormality in urine volume, insomnia, and sleep disorder.

### MODE FOR CARRYING OUT THE INVENTION

The term “life force (qi)” refers to energy of the living body, and it is considered that the “life force” is involved in a function of a signal transduction system of the living body, mainly such as a nervous system, or an endocrine system. The term “qi deficiency” refers to a condition in which the energy of the living body is insufficient. The “qi deficiency” is a condition of being weak, lacking in energy, and being unable to be active, and the “qi deficiency” is considered to be caused based on the decline in gastrointestinal function and digestive and absorption function. Examples of the symptoms relating to the qi deficiency include chronic fatigue, fatigability, malaise, weakness, listlessness, mental instability, diarrhea, abnormality in urine volume, frequent urination, enuresis, urinary incontinence, nocturnal enuresis, daytime sleepiness, anorexia, susceptibility to infection, hypoplasia, feeling of cold, cold extremity, swelling, dyspepsia, thinness, dry skin, excessive sweating, and friability.

The “blood deficiency” is considered to be a condition in which a function of a blood/immune system is weakened. Examples of the symptoms relating to the blood deficiency include anemia, immunological deterioration, poor concentration, insomnia, sleep disorder, hair coming off easily, visual fatigue, dryness and rough of skin, abnormality of nails, cramp, paresthesia, oligomenorrhea, menstrual irregularity, cold extremity, fatigability, susceptibility to infection, and ill complexion.

In the present specification, the symptoms (lesions or physical disorders) relating to qi deficiency and/or blood deficiency are mainly symptoms relating to qi deficiency, and further refer to various symptoms relating to blood deficiency brought about by the qi deficiency. Specific examples of the symptoms relating to qi deficiency and/or blood deficiency include at least one selected from the group consisting of chronic fatigue, fatigability, malaise, listlessness, abnormality in urine volume, insomnia, and sleep disorder, preferably include chronic fatigue, fatigability, malaise, listlessness, insomnia, and sleep disorder, and particularly preferably include chronic fatigue, fatigability, malaise, and listlessness. In this regard, the term “abnormality in urine volume” refers to a small or large urine volume, and particularly refers to a small urine volume. The symptoms relating to qi deficiency and/or blood deficiency may further include weakness, diarrhea, frequent urination, anorexia, urinary incontinence, cold extremity, feeling of cold, susceptibility to infection, excessive sweating, ill complexion, and the like.

The various symptoms relating to qi deficiency and/or blood deficiency may also be caused by a cause other than the qi deficiency and/or the blood deficiency. For example, a symptom such as diarrhea may be caused by bacteria, poisons, drugs, or the like, and further, a symptom such as malaise may be caused by transient muscle pain, excessive fatigue, undernutrition, overeating, or the like. However, in such a case, once these specific causes are eliminated, the symptoms usually disappear. On the other hand, the qi deficiency and/or the blood deficiency are often a chronic symptom because they are related to a lack of “life force” and/or “blood”, and there is often no specific obvious cause. Accordingly, at least one (for example, at least two, at least three, at least four, at least five, at least six, or at least seven) of the various symptoms relating to qi deficiency and/or blood deficiency is likely to be symptoms relating to qi deficiency and/or blood deficiency if the symptoms continue over a period of one or more weeks, two or more weeks, four or more weeks, three or more months, six or more months, or one or more years, and is more likely to be the qi deficiency and/or the blood deficiency if there is no direct cause for these symptoms. More reliably, such symptoms can be diagnosed as the qi deficiency and/or the blood deficiency based on Chinese medicine.

The composition of the present embodiment includes components (1), (2), (3), and (4) as the active components. The components (1), (2), (3), and (4) each contain a group of herbal medicines having specific medicinal benefit coverages, and nature tastes. The medicinal benefit coverage, nature taste, and efficacy of each herbal medicine are as described later. In addition, for each herbal medicine, the generally known origin and site may be used, and the details are as described later.

In this regard, the medicinal benefit coverage is one of the concepts used in Chinese medicine (TCM), refers to which part of the human body a herbal medicine acts on, and indicates the action site where the medicinal effect appears. The medicinal benefit coverage is composed of so-called “five organs”, that is, “liver/heart/spleen/lung/kidney”, and the five organs in Chinese medicine do not correspond to anatomical organs, but rather mean “functions” of the human body. The liver is related to the autonomic nerve, the emotion, the motor nerve, the gynecological system, and the liver, stores the blood, and controls the blood so that the life force and blood smoothly flow. The heart is related to the brain and the circulatory system including the heart, and has a function of circulating the blood throughout the body and nourishing the body. The spleen is related to the digestive system, allergies, and part of water metabolism, digests and absorbs foods and drinks, and creates “life force, blood, and colorless bodily fluids”, which are the basics of the body. The lung is related to the respiratory system, the skin, part of water metabolism, and allergies, works together with the spleen to create “life force”, spreads the life force throughout the body, regulates the flow of colorless bodily fluids, protects the body from external bad vibes, and is closely related to the immune system. The kidney is related to the reproductive system, the urinary system, the endocrine system, and aging and development, stores the source of vital energy, controls the water metabolism of the whole body, and produces and reabsorbs urine. In Chinese medicine, the five organs work together to generate and store the vital spirit of the body, and control the mental activity.

The nature taste (also referred to as “qi taste”) is a type of classification system that indicates a nature of a herbal medicine, each taste is related to the five organs, and the taste itself has its own role. Most commonly, the nature taste indicates a nature of cooling and warming the human body by five natures of “cold/cool/flat/warm/heat”, indicates a taste at the time of ingestion by five tastes of “hot/sweet/acid/bitter/salty”, and is also an indicator of the efficacy. In the five tastes, “light/astringent” may also be added in some cases. The “heat” and “warm” have a function of warding off cold, warming the body, stimulating the body, and activating the body. In addition, the “cold” and “cool” have a function of cooling the body heat, detoxifying, and calming the mind, and the “flat” refers to a nature of neither warming nor cooling. The “cold” may be further divided into “great cold”, “cold”, and “slight cold” depending on the degree. The “hot” is related to the lung, and has an effect of sweating, circulating the life force, and diverting oneself. The “sweet” is related to the spleen, and has a supplementing or alleviating effect. The “acid” is related to the liver, and has a tightening effect. The “bitter” is related to the heart, and has an effect of cooling heat, and drying and lowering dampness. The “salty” is related to the kidney, and has an effect of softening hard things, and moistening and lowering them. The nature taste may be subdivided into “great cold”, “slight bitter” and the like depending on the degree.

The medicinal benefit coverage and nature taste are set for almost all the herbal medicines, herbal medicines that are coincident in the medicinal benefit coverage and/or the nature taste tend to have the same efficacy. The medicinal benefit coverage and nature taste are representatives of technical ideas that help to systematize the efficacy/prescription design of a huge number of herbal medicines.

In addition, as the classification of herbal medicines, a formula for relieving superficies syndrome with pungent and cool natured drugs, a formula for clearing heat-fire, an emetic, a cathartic, a formula for diuresis and diffusing dampness, a formula for dispelling wind and eliminating dampness, a formula for dispelling cold, a formula for clearing heat, an antitussive and expectorant combination, a formula for regulating qi-flowing, a formula for invigorating qi, a formula for regulating blood condition, a formula for nourishing blood, a formula for promoting blood circulation and removing blood stasis, a formula for taking a tonic to build up health, a formula for opening pores with aromatic, a sedative-antispasmodic, an astringent, a digestant, an anthelmintic, a topical agent, and the like can be mentioned.

### Component (1)

Component (1) is a herbal medicine having a medicinal benefit coverage of “spleen”, and specifically at least one kind selected from the group consisting of honey-fried licorice root, licorice root, Dioscorea japonica Thunberg, jujube fruit, malt starch syrup, and ginseng root. Among these herbal medicines, the component (1) is preferably at least one kind selected from the group consisting of honey-fried licorice root, licorice root, Dioscorea japonica Thunberg, jujube fruit, and malt starch syrup from the viewpoint of availability (supply stability), and more preferably at least one kind selected from the group consisting of honey-fried licorice root, licorice root, and jujube fruit from the viewpoint of exerting an effect with a small amount of raw material herbal medicines. The component (1) may be at least one kind of herbal medicine, at least two kinds of herbal medicines, at least three kinds of herbal medicines, or at least four kinds of herbal medicines.

Honey-fried licorice root is dried root and stolons of Glycyrrhiza uralensis (Glycyrrhiza uralensis Fisch.) or Liquorice (Glycyrrhiza glabra L.) roasted as it is or with coating with honey, the nature taste is flat, and sweet, the medicinal benefit coverage is twelve meridians, and the efficacy is invigorating the spleen-stomach and replenishing qi-flowing, moistening the lung/eliminating phlegm and relieving cough, relieving pain according to the urgency, clearing heat and removing toxicity, and harmonizing properties of medicine. The main components are triterpene glycosides, and flavanone glycosides.

Licorice root is dried root and stolons of Glycyrrhiza uralensis (Glycyrrhiza uralensis Fisch.) or Liquorice (Glycyrrhiza glabra L.), the nature taste is flat, and sweet, the medicinal benefit coverage is twelve meridians, and the efficacy is invigorating the spleen-stomach and replenishing qi-flowing, moistening the lung/eliminating phlegm and relieving cough, relieving pain according to the urgency, clearing heat and removing toxicity, and harmonizing properties of medicine. The main components are triterpene glycosides, and flavanone glycosides.

Dioscorea japonica Thunberg is a dried rhizome (rhizophore) excluding the periderm of Japanese yam (Dioscorea japonica Thunb.) or Chinese yam (Dioscorea batatas Decne.), the nature taste is flat, and sweet, the medicinal benefit coverage is spleen/lung/kidney, and the efficacy is invigorating the spleen and relieving diarrhea, nourishing yin and supporting the spleen, nourishing the lung and benefiting yin/relieving cough, and tonifying the kidney and consolidating the essence/reducing urination/arresting leucorrhea. The main components are polysaccharides (starch, and glycoprotein).

Jujube fruit is a dried fruit of jujube (Zizyphus jujuba Mill. var. inermis (Bunge) Rehder), the nature taste is slight warm, and sweet, the medicinal benefit coverage is spleen/stomach/heart/liver, and the efficacy is invigorating the spleen and harmonizing the stomach, nourishing the body and soothing the nerves, and moderating properties of medicine. The main components are fructose, and glucose.

Malt starch syrup is made by hydrolyzing and saccharifying starch of maize (Zea mays L.), cassava (Manihot esculenta Crantz), potato (Solanum tuberosum L.), sweet potato (Ipomoea batatas (L.) Poir.), or rice plant (Oryza sativa L.), or seeds excluding the seed coat of rice plant, the nature taste is slight warm, and sweet, the medicinal benefit coverage is spleen/stomach/lung, and the efficacy is replenishing deficiency and invigorating the spleen-stomach/relieving pain according to the urgency, and moistening the lung and relieving cough. The main component is maltose.

Ginseng root is a root excluding thin roots of or a dried root excluding thin roots made by lightly blanching and drying a root of Panax ginseng (Panax ginseng C.A. Mey.), the nature taste is slight warm, and sweet/slight bitter, the medicinal benefit coverage is lung/spleen, and the efficacy is invigorating qi for relieving desertion, invigorating spleen qi, restoring lung qi, and promoting fluid production for quenching thirst. The main components are saponin, and flavonoid.

### Component (2)

Component (2) is a herbal medicine having a medicinal benefit coverage of “liver” or “spleen”, and is specifically at least one kind selected from the group consisting of wild turmeric, longan aril, zedoary root, Suberect Spatholobus stem, Luffa vegetable sponge, Sparganium rhizome, safflower, and Artemisia anomala. Among these herbal medicines, the component (2) is preferably at least one kind selected from the group consisting of wild turmeric, longan aril, zedoary root, Suberect Spatholobus stem, Luffa vegetable sponge, and safflower from the viewpoint of availability (supply stability), more preferably at least one kind selected from the group consisting of wild turmeric, zedoary root, Suberect Spatholobus stem, and safflower from the viewpoint of the efficacy, and particularly preferably at least one kind selected from the group consisting of wild turmeric, zedoary root, and Suberect Spatholobus stem. The component (2) may be at least one kind of herbal medicine, at least two kinds of herbal medicines, at least three kinds of herbal medicines, or at least four kinds of herbal medicines.

Wild turmeric is a dried rhizome made usually by blanching and drying a rhizome of turmeric (Curcuma longa L.) as it is or after removing the cork layer, the nature taste is warm, and bitter/hot, the medicinal benefit coverage is spleen/liver, and the efficacy is breaking blood circulation and activating qi flowing/restoring menstrual flow and relieving pain, and dispelling wind and overcoming dampness. The main components are essential oil, polyphenol, and starch.

longan aril is a dried aril of longan (Euphoria longana Lam), the nature taste is flat, and sweet, the medicinal benefit coverage is heart/spleen, and the efficacy is nourishing the heart and blood, tranquillization, and invigorating spleen qi. The main components are saccharides, and organic acid.

Zedoary root is a dried rhizome made usually by blanching and drying a rhizome of zedoary (Curcuma zedoaria Roscoe, Curcuma phaeocaulis Valeton, Curcuma kwangsiensis S. G. Lee et C. F. Liang), the nature taste is warm, and bitter/hot, the medicinal benefit coverage is liver/spleen, and the efficacy is activating qi flowing and breaking blood, and eliminating accumulation and relieving pain. The main components are essential oil, polyphenol, and starch.

Suberect Spatholobus stem is a dried climbing stem of Spatholobus suberectus Dunn, the nature taste is warm, and bitter/slight sweet, the medicinal benefit coverage is liver/kidney, and the efficacy is promoting blood circulation and replenishing blood, and relieving rigidity of muscle for removing obstruction in collaterals. The main components are a phenolic compound, iron, and an amino acid.

Luffa vegetable sponge is a dried vascular bundle of ripe fruit of loofah (Luffa cylindrica (L.) M. Roem.), the nature taste is flat, and sweet, the medicinal benefit coverage is lung/stomach/liver, and the efficacy is activating blood circulation for removing obstruction in collaterals. The main components are polysaccharides, and a phenolic compound.

Sparganium rhizome is a dried tuber excluding the outer skin of bur reed (Sparganium stoloniferum (Graebn.) Buch.-Ham. ex Juz.), the nature taste is flat, and bitter, the medicinal benefit coverage is liver/spleen, and the efficacy is breaking blood and activating qi flowing, and eliminating accumulation and relieving pain. The main components are essential oil, and starch.

Safflower is a tubular flower as it is or a tubular flower after removing most of the yellow pigment made sometimes by pressing into a plate shape of Carthamus tinctorius L., the nature taste is warm, and hot, the medicinal benefit coverage is heart/liver, and the efficacy is promoting blood circulation and restoring menstrual flow, and eliminating blood stasis and relieving pain. The main component is flavonoid.

Artemisia anomala is a dried whole grass of Artemisia anomala S. Moore, the nature taste is warm, and hot/bitter, the medicinal benefit coverage is heart/liver/spleen/lung/kidney/bladder, and the efficacy is breaking blood circulation and restoring menstrual flow/removing distension and relieving pain, hemostasis for removing edema, clearing heat and diuresis, and lowering qi and removing distension. The main component is essential oil.

### Component (3)

Component (3) is a herbal medicine having a medicinal benefit coverage of “lung”, and a nature taste of “cool”, “cold”, or “slight cold”, and is specifically at least one kind selected from the group consisting of tea leaf, mentha herb, mulberry leaf, chrysanthemum flower, and Koushi. Among these herbal medicines, the component (3) is preferably at least one kind selected from the group consisting of tea leaf, mentha herb, and mulberry leaf from the viewpoint of exerting an effect with a small amount of raw material herbal medicine, and more preferably at least one kind selected from the group consisting of tea leaf, and mulberry leaf from the viewpoint of the flavor. The component (3) may be at least one kind of herbal medicine, at least two kinds of herbal medicines, at least three kinds of herbal medicines, or at least four kinds of herbal medicines.

Tea leaf is a dried leaf (often with a branch tip) of tea (Camellia sinensis (L.) Kuntze), the nature taste is slight cold, and bitter/slight sweet, the medicinal benefit coverage is heart/lung/liver/kidney/spleen/stomach, and the efficacy is dispelling wind/refreshing the head and eyes, clearing heat and lowering fire/resolving summer-heat, reducing fever and toxicity/relieving dysentery, and diuresis. The main components are alkaloid, flavonoid, and tannin.

Mentha herb is a dried aerial part of Mentha arvensis L. var. piperascens Malinv., the nature taste is cool, and hot, the medicinal benefit coverage is lung/liver, and the efficacy is dispelling wind and heat, clearing the head and eyes/soothing the pharynx, promoting eruption and relieve itching, dispersing stagnated liver qi and relieving depression, and dispelling. The main component is essential oil.

Mulberry leaf is a dried leaf of Morus alba L., the nature taste is cold, and bitter/sweet, the medicinal benefit coverage is lung/liver, and the efficacy is dispelling wind and heat, clearing lung-heat and moistening dryness/relieving cough, calming liver yang, and improving eyesight. The main components are flavonoid, and mineral.

Chrysanthemum flower is a dried flower head of Chrysanthemum morifolium Ramat. or Chrysanthemum indicum L., the nature taste is cool, and sweet/slight bitter, the medicinal benefit coverage is lung/liver, and the efficacy is dispelling wind and heat, improving eyesight, and calming liver yang. The main components are flavonoid, and sesquiterpene.

Koushi is a dried ripe seed made by steaming and fermenting a ripe seed of soy (Glycine max (L.) Merr. subsp. Max), the nature taste is cool, and hot/sweet/slight salty, the medicinal benefit coverage is lung/stomach, and the efficacy is dispersing and resolving the superficies, and draining depression and eliminating agitation. The main components are fat, and a protein.

### Component (4)

Component (4) is a herbal medicine having a medicinal benefit coverage of “kidney”, “spleen”, or “stomach”, and is specifically at least two kinds selected from the group consisting of ripe tangerine peel, Psoralea corylifolia fruit, Chaenomeles speciosa fruit, Piper longum, cinnamon twig, Perilla, ginger root, coriander, Allium fistulosum Linne, Zizyphi Spinosi Semen, Chinese arborvitae kernel, plantago seed, plantago herb, coix seed, corn silk, belvedere Fruit, Benincasa seed, knotgrass, snowbellleaf tickclover herb, cinnamon, processed ginger, fennel, Zanthoxylum fruit, Piper nigrum, Eleutherococcus Senticosus Rhizome, and Artemisia leaf. The component (4) is a herbal medicine that generates life force, and has an effect of warming the middle energizer relating to resistance/frailty. When only one kind of a herbal medicine to constitute the component (4) is mixed, the effect of warming the middle energizer is limited and antagonizes the component (3), and the effect of the present invention cannot be sufficiently exerted, but when two kinds of herbal medicines to constitute the component (4) are mixed, the middle energizer can be sufficiently warmed, and it can be expected to act synergistically with the actions of other components and to exert the effect of the present invention. For your reference, in Chinese medicine, the middle energizer is one of the triple energizer regions, and the triple energizer regions are considered to be not an organ in a single form but one functional unit, and is classified into “upper energizer” that refers to the chest above the diaphragm, “middle energizer” that refers to the abdomen between the diaphragm and the navel, and “lower energizer” that refers to the part below the stomach. The middle energizer, which is triple energizer, includes the spleen and the stomach, is involved in the digestion and transportation of nutrients to generate life force, is a source of resistance, and further is related to frailty.

Among these herbal medicines, the component (4) is from the viewpoint of availability (supply stability), preferably at least two kinds selected from the group consisting of ripe tangerine peel, Psoralea corylifolia fruit, Chaenomeles speciosa fruit, Piper longum, cinnamon twig, Perilla, ginger root, coriander, plantago seed, plantago herb, coix seed, corn silk, belvedere Fruit, cinnamon, processed ginger, fennel, and Eleutherococcus senticosus Rhizome, and is from the viewpoint of the efficacy, more preferably at least two kinds selected from the group consisting of ripe tangerine peel, Psoralea corylifolia fruit, Chaenomeles speciosa fruit, Piper longum, cinnamon twig, Perilla, ginger root, coriander, cinnamon, processed ginger, fennel, and Eleutherococcus senticosus Rhizome, and particularly preferably at least two kinds selected from the group consisting of Psoralea corylifolia fruit, Piper longum, cinnamon, processed ginger, and Eleutherococcus Senticosus Rhizome. The component (4) may be at least two kinds of herbal medicines, at least three kinds of herbal medicines, at least four kinds of herbal medicines, or at least five kinds of herbal medicines.

Further, from the viewpoint of ease of commercialization processing, the component (4) is preferably at least two kinds (for example, at least three kinds, at least four kinds, or at least five kinds of herbal medicines) selected from the group consisting of ripe tangerine peel, Psoralea corylifolia fruit, Chaenomeles speciosa fruit, Piper longum, cinnamon twig, Perilla, coriander, plantago herb, coix seed, corn silk, belvedere Fruit, cinnamon, fennel, and Eleutherococcus senticosus Rhizome.

Ripe tangerine peel is a formula for activating qi flowing, and is a dried ripe pericarp of Satsuma mandarin (Citrus unshiu (Swingle) Marcow.) or Ponkan mandarin (Citrus reticulata blanco), the nature taste is warm, and hot/bitter, the medicinal benefit coverage is spleen/lung, and the efficacy is regulating qi-flowing for invigorating the spleen, and drying dampness and dissipating phlegm. The main components are essential oil, and flavonoid.

Psoralea corylifolia fruit is a formula for reinforcing yang, and is a dried ripe fruit of breadroot (Psoralea corylifolia L.), the nature taste is great warm, and hot/bitter/astringent, the medicinal benefit coverage is kidney/spleen, and the efficacy is tonifying the kidney and strengthening yang-qi/consolidating the essence and reducing urination, warming the spleen and relieving diarrhea, and warming the kidney and improving inspiration/calming panting. The main components are essential oil, an organic acid, and flavonoid.

Chaenomeles speciosa fruit is a formula for dispelling wind and eliminating dampness, and is a dried near-ripe or ripe pseudocarp of Chinese quince (Chaenomeles sinensis (thouin) Koehne) or Japanese quince (Chaenomeles speciosa (Sweet) Nakai), the nature taste is warm, and acid, the medicinal benefit coverage is liver/spleen, and the efficacy is calming the liver and relieving rigidity of muscle, and harmonizing the spleen-stomach and removing dampness. The main components are saponin, an organic acid, and tannin.

Piper longum is a formula for dispelling cold, and is a dried ear of near-ripe or ripe fruit of long pepper (Piper longum L.), the nature taste is heat, and hot, the medicinal benefit coverage is stomach/large intestine, and the efficacy is warming the spleen-stomach for dispelling cold/activating qi flowing and relieving pain. The main components are essential oil, and alkaloid.

Cinnamon twig is a formula for relieving superficies syndrome with pungent and warm natured drugs, and is a dried twig of Cinnamomum cassia (Cinnamomum cassia Blume), the nature taste is warm, and hot/sweet, the medicinal benefit coverage is lung/heart/spleen/liver/kidney/bladder, and the efficacy is sweating and expelling pathogenic factors from muscles, warmly dredging for meridian flow, stimulating yang for transforming qi, and equilibrium for descending adverse qi. The main components are essential oil, and tannin.

Perilla is a formula for relieving superficies syndrome with pungent and warm natured drugs, and is a dried leaf and branch of Perilla frutescens (L.) Britton var. crispa (Benth.) W. Deane, the nature taste is warm, and hot, the medicinal benefit coverage is lung/spleen/stomach, and the efficacy is dispelling cold and resolving the superficies, regulating qi-flowing and relaxing the spleen-stomach, activating qi flowing and tranquilizing fetus to prevent miscarriage, and resolving fish and crab poison. The main components are essential oil, anthocyanin glycosides, and flavonoid.

Ginger root is a formula for relieving superficies syndrome with pungent and warm natured drugs, and is a dried rhizome made by drying a rhizome whose periderm has been sometimes removed of ginger (Zingiber officinale Roscoe), the nature taste is slight warm, and hot, the medicinal benefit coverage is lung/spleen/stomach, and the efficacy is dispelling cold and resolving the superficies, warming the stomach and relieving vomiting, dissipating phlegm for moving water, and removing toxicity. The main components are essential oil, a hot taste component, and starch.

Coriander is a formula for relieving superficies syndrome with pungent and warm natured drugs, and is a dried whole grass of cilantro (Coriandrum sativum L.), the nature taste is warm, and hot, the medicinal benefit coverage is lung/stomach, and the efficacy is resolving superficies and promoting eruption, and stimulating appetite and helping to digest. The main components are essential oil, and terpene.

Allium fistulosum Linne is a formula for relieving superficies syndrome with pungent and warm natured drugs, and is a dried bulb of green onion (Allium fistulosum L. 9), the nature taste is warm, and hot, the medicinal benefit coverage is lung/stomach, and the efficacy is dispelling cold and resolving the superficies, and stimulating yang for dispelling cold. The main components are essential oil, and a sulfur-containing compound.

Zizyphi Spinosi Semen is a formula for nourishing the heart and soothing the nerves, and is a dried ripe seed of Chinese date (Ziziphus jujuba Mill. var. spinosa (Bunge) Hu ex H.F. Chow), the nature taste is flat, and sweet/acid, the medicinal benefit coverage is heart/liver/bile/spleen, and the efficacy is replenishing the liver and tranquillizing the heart, and convergence for arresting sweating. The main components are triterpene, saponin, and flavonoid.

Chinese arborvitae kernel is a formula for nourishing the heart and soothing the nerves, and is a dried ripe seed excluding the seed coat of oriental arbor-vita (Platycladus orientalis (L.) Franco), the nature taste is flat, and sweet, the medicinal benefit coverage is heart/spleen/liver/kidney, and the efficacy is nourishing the heart and soothing the nerves, benefiting yin and arresting sweating, and benefiting the spleen and clearing the intestine. The main components are fatty oil, and saponin.

Plantago seed is a formula for diuresis and diffusing dampness, and is a dried ripe seed of plantain (Plantago asiatica L.), the nature taste is cold, and sweet/light, the medicinal benefit coverage is liver/kidney/lung/small intestine, and the efficacy is clearing heat and promoting urination, diffusing dampness and relieving diarrhea, removing liver-fire for improving eyesight, and dissipating phlegm and relieving cough. The main components are mucilaginous polysaccharide, and iridoid glucosides.

Plantago herb is a formula for diuresis and diffusing dampness, and is a dried whole grass in flowering time of plantain (Plantago asiatica L.), the nature taste is cold, and sweet/light, the medicinal benefit coverage is liver/kidney/lung/small intestine, and the efficacy is clearing heat and promoting urination, diffusing dampness and relieving diarrhea, removing liver-fire for improving eyesight, and dissipating phlegm and relieving cough. The main components are iridoid glucosides, and flavonoid.

Coix seed is a formula for diuresis and diffusing dampness, and is a dried ripe seed excluding the seed coat of adlay (Coix lacryma-jobi var.ma-yuen (Rom.Caill.) Stapf), the nature taste is slight cold, and sweet/light, the medicinal benefit coverage is spleen/stomach/lung, and the efficacy is clearing and disinhibiting dampness-heat, removing dampness and relieving numbness, expelling pus and removing edema, and invigorating the spleen and relieving diarrhea. The main components are starch, and a protein.

Corn silk is a formula for diuresis and diffusing dampness, and is a dried style of maize (Zea mays L.), the nature taste is flat, and sweet, the medicinal benefit coverage is liver/kidney/bladder/heart/small intestine, and the efficacy is inducing diuresis and removing edema, and curing jaundice. The main components are saponin, and flavonoid.

belvedere Fruit is a formula for diuresis and diffusing dampness, and is a dried ripe fruit of Kochia (Kochia scoparia (L.) Schrad.), the nature taste is cold, and hot/bitter, the medicinal benefit coverage is kidney/bladder, and the efficacy is clearing dampness and heat/facilitating urination, and dispelling wind and relieving itching. The main components is triterpenoid saponin.

Benincasa seed is a formula for diuresis and diffusing dampness, and is a dried ripe seed of wax gourd (Benincasa cerifera savi) or Benincasa cerifera savi forma emarginata K. Kimura et Sugiyama, the nature taste is slight cold, and sweet, the medicinal benefit coverage is lung/stomach/large intestine/small intestine, and the efficacy is clearing heat/inducing diuresis and removing edema. The main components are a fatty acid, and alkaloid.

Knotgrass is a formula for diuresis and diffusing dampnes, and is a dried aerial part of prostrate knotweed (Polygonum aviculare L.), the nature taste is flat, and bitter, the medicinal benefit coverage is stomach/bladder, and the efficacy is promoting diuresis for stranguria, and killing insects and relieve itching. The main components are flavone glycosides, and tannin.

Snowbellleaf tickclover herb is a formula for diuresis and diffusing dampness, and is a dried whole grass of Lysimachia christinae Hance, the nature taste is slight cold, and sweet/salty/light, the medicinal benefit coverage is liver/bile/kidney/bladder, and the efficacy is promoting diuresis for stranguria/expelling stones and relieving pain, and clearing heat and removing dampness/curing jaundice. The main components are essential oil, and tannin.

Cinnamon is a formula for dispelling cold, and is a dried bark or bark excluding part of the periderm of Cinnamomum cassia (Cinnamomum cassia Blume), the nature taste is great heat, and hot/sweet, the medicinal benefit coverage is liver/kidney/heart/spleen/stomach, and the efficacy is warming the spleen-stomach and tonifying yang, dispelling cold and relieving pain, and warmly dredging for meridian flow. The main components are essential oil, and tannin.

Processed ginger is a formula for dispelling cold, and is a dried rhizome made by blanching or steaming a rhizome of ginger (Zingiber officinale Roscoe), the nature taste is great heat, and great hot, the medicinal benefit coverage is heart/lung/spleen/stomach, and the efficacy is warming the spleen-stomach for dispelling cold, restoring yang for restoring flow, and warming the lung and dissipating phlegm/resolving fluid retention. The main components are essential oil, a hot taste component, and starch.

Fennel is a formula for dispelling cold, and is a dried ripe fruit of fennel (Foeniculum vulgare Mill.), the nature taste is warm, and hot, the medicinal benefit coverage is liver/kidney/spleen/stomach, and the efficacy is dispelling cold and relieving pain, and regulating qi-flowing and harmonizing the stomach. The main component is essential oil.

Zanthoxylum fruit is a formula for dispelling cold, and is a dried ripe pericarp whose seeds have been removed as much as possible of Sichuan pepper (Zanthoxylum bungeanum Maxim.) or winged prickly ash (Zanthoxylum armatum DC. var. subtrifoliatum (Franch.) Kitam.), the nature taste is heart, and hot, the medicinal benefit coverage is spleen/stomach/kidney, and the efficacy is dispelling cold and relieving pain/drying dampness, and removing toxicity and exterminating insects. The main components are essential oil, and a hot taste component.

Piper nigrum is a formula for dispelling cold, and is a dried near-ripe or ripe fruit of Piper nigrum L., the nature taste is heart, and hot, the medicinal benefit coverage is stomach/large intestine, and the efficacy is warming the spleen-stomach for dispelling cold/activating qi flowing and relieving pain. The main components are essential oil, and alkaloid.

Eleutherococcus Senticosus Rhizome is a formula for dispelling wind and eliminating dampness, and is a dried root or rhizome of Siberian ginseng (Eleutherococcus senticosus (Rupr.et Maxim.) Maxim.), the nature taste is warm, and hot/bitter, the medicinal benefit coverage is liver/kidney, and the efficacy is relieving rheumatism, replenishing the liver and kidney/strengthening the muscles and bones, and inducing diuresis and removing edema. The main components are saponin, and lignan.

Artemisia leaf is a formula for dispelling cold, and is a dried leaf and branch of Japanese mugwort (Artemisia princeps Pamp.) or Artemisia montana (Artemisia montana (Nakai) Pamp.), the nature taste is warm, and bitter/hot, the medicinal benefit coverage is liver/spleen/kidney, and the efficacy is dispelling cold and removing dampness/relieving pain, and warming meridian and relieving pain. The main components are essential oil, a fatty acid, and tannin.

The herbal medicines as the components (1), (2), (3), and (4), which are contained in the composition of the present embodiment may be each a herbal medicine as it is, may be each a herbal medicine finely shredded or crushed, or may be each an extract of the herbal medicine. In the present specification, the extract of a herbal medicine is preferably an extract obtained by extracting the herbal medicine with a pharmaceutically acceptable aqueous solvent such as water (including hot water), ethanol, or a mixed solvent of water and ethanol. The herbal medicine extract may be prepared by a known extraction method, or may be a general soft extract, or an extract powder/extract granules.

The mixing amounts of herbal medicines as the components (1), (2), (3), and (4) contained in the composition of the present embodiment may be each appropriately set according to various conditions of the person who ingests the composition, such as body weight, age, and symptoms, may be each an amount sufficient to prevent, improve, or alleviate symptoms relating to qi deficiency and/or blood deficiency, and are each prescribed by the weight of herbal medicine per daily dose (in a case of an extract, the weight of herbal medicine used (herbal medicine equivalent weight)) (g/day).

As the daily dose of herbal medicine, the component (1) includes 3 to 6 g of honey-fried licorice root, 3 to 6 g of licorice root, 9 to 30 g of Dioscorea japonica Thunberg, 3 to 9 g of jujube fruit, 30 to 60 g of malt starch syrup, and 3 to 9 g of ginseng root. As the daily dose of herbal medicine, the component (2) includes 3 to 9 g of wild turmeric, 6 to 12 g of longan aril, 3 to 9 g of zedoary root, 9 to 15 g of Suberect Spatholobus stem, 6 to 15 g of Luffa vegetable sponge, 3 to 9 g of Sparganium rhizome, 3 to 9 g of safflower, and 9 to 15 g of Artemisia anomala. As the daily dose of herbal medicine, the component (3) includes 3 to 15 g of tea leaf, 1.5 to 6 g of mentha herb, 3 to 9 g of mulberry leaf, 6 to 12 g of chrysanthemum flower, and 9 to 15 g of Koushi. As the daily dose of herbal medicine, the component (4) includes 3 to 9 g of ripe tangerine peel, 3 to 9 g of Psoralea corylifolia fruit, 3 to 9 g of Chaenomeles speciosa fruit, 1.5 to 6 g of Piper longum, 3 to 10 g of cinnamon twig, 6 to 12 g of Perilla, 3 to 9 g of ginger root, 3 to 6 g of coriander, 3 to 9 g of Allium fistulosum Linne, 9 to 15 g of Zizyphi Spinosi Semen, 6 to 15 g of Chinese arborvitae kernel, 3 to 9 g of plantago seed, 3 to 9 g of plantago herb, 9 to 30 g of coix seed, 15 to 30 g of corn silk, 9 to 15 g of belvedere Fruit, 15 to 30 g of Benincasa seed, 9 to 30 g of knotgrass, 15 to 30 g of snowbellleaf tickclover herb, 1.5 to 5 g of cinnamon, 3 to 9 g of processed ginger, 3 to 9 g of fennel, 3 to 6 g of Zanthoxylum fruit, 1.5 to 3 g of Piper nigrum, 3 to 9 g of Eleutherococcus Senticosus Rhizome, and 3 to 6 g of Artemisia leaf. In a case where multiple herbal medicines are used as the component (1), (2), (3), or (4), the daily dose of each herbal medicine may be appropriately reduced.

The composition of the present embodiment is a composition for use in preventing, improving, or alleviating symptoms relating to qi deficiency and/or blood deficiency. The composition of the present embodiment intends to supplement the blood deficiency by generating and supplementing life force by allowing the component (1) to act on the spleen, and further by helping the component (2) to make and circulate blood in addition to the functioning of the life force supplemented. In addition, the component (4) generates life force, and enhances an action of supplementing the life force by warming the middle energizer relating to resistance/frailty, and further the component (3) appropriately balances the cold/warm, and synergistically exerts an ataractic tranquilizing effect. As described above, by containing the components (1), (2), (3), and (4), various symptoms relating to qi deficiency and/or blood deficiency can be prevented, improved, or alleviated.

The symptoms relating to qi deficiency and/or blood deficiency are at least one, at least two, at least three, at least four, or at least five selected from the group consisting of chronic fatigue, fatigability, malaise, listlessness, abnormality in urine volume, insomnia, and sleep disorder, and in particular, preferably at least one, at least two, at least three, at least four, or at least five selected from the group consisting of chronic fatigue, fatigability, malaise, listlessness, insomnia, and sleep disorder. In addition, at least one of the symptoms preferably continues over a period of one or more weeks, two or more weeks, four or more weeks, three or more months, six or more months, or one or more years.

The composition of the present embodiment is preferably a food composition or a pharmaceutical composition. The herbal medicines to be contained in the composition of the present embodiment are not recorded in herbal medicines and the like of The Pharmacopoeia of Japan and not regulated for use, and thus, can be used for both medical care and foods. Accordingly, the composition of the present embodiment can be a food composition such as a functional food. Therefore, there is an advantage that discomfort symptoms and physical disorders can be easily prevented, improved, or alleviated without requiring any prescription by a doctor.

The food composition may be in any possible form as food. Examples of the form include: a milk product; a fermented food (yogurt, cheese, et al.); beverages (coffee, juice, a soft drink such as a tea beverage, a carbonated beverage, a milk beverage, a lactic acid bacteria beverage, a beverage containing lactic acid bacteria, a yogurt drink, and liquors such as Japanese rice wine, fruit wine, and Western liquor); spreads (custard cream, et al.); pastes (fruit paste, et al.); Western confectionery (donuts, pies, cream puffs, chewing gum, candy, jelly, cookies, cake, chocolate, pudding, et al.); Japanese confectionery (Daifuku (soft round rice cake stuffed with sweet bean jam), rice cake, sweet bun (bun with a bean-jam filling), sponge cake, Anmitsu (agar jelly served with red bean paste and brown sugar syrup), Yokan (sweet bean jelly), et al.); ice confectionery (ice cream, popsicles, sherbet, et al.); foods (curry, Gyudon (a bowl of rice topped with boiled beef and onions), porridge of rice and vegetables, rice gruel, miso (fermented soybean paste) soup, soup, meat sauce, pasta, Japanese pickled vegetables, jam, etc.); and condiments (dressing, flavor enhancer, Furikake (dried food sprinkling over rice), broth, et al.). Such a food composition can be served as a dish at a restaurant, and can also be provided in a form of general distribution by taking a form of prepared food, retort pouch food, a form of frozen food, another packaging form or the like of a general food composition.

In addition to the components (1), (2), (3), and (4) as the active components, the food compositions in the above various forms may contain a component that is acceptable as food, as long as it does not affect the action of the active components. Examples of the component include various general foodstuffs, starch, gelatin, vegetable fat and oil, animal fat and oil, processed fat and oil or the like, carboxymethyl cellulose, gum arabic, locust bean gum, gum tragacanth, karaya gum, konjac glucomannan, xanthane gum, agar, carrageenan, polyacrylic acid, carob bean gum, alginic acid, a gellant such as a cyclodextrin, a seasoning, a sweetener, a spice, a colorant, a preservative, an antioxidant, water, an algefacient, a binder, a sweetener, a disintegrant, a lubricant, a colorant, flavor, a stabilizer, an antiseptic, a sustained release regulator, a surfactant, a solubilizer, and a wetting agent.

The food composition of the present embodiment may appropriately arbitrarily mix a functional food material for the purpose of further exerting a complex effect. As the functional food material, for example, various kinds of herbal medicines other than the components (1), (2), (3), and (4), various kinds of probiotics, various kinds of oligosaccharides, nutrient components such as vitamins and minerals, flavonoids, quinones, polyphenols, amino acid, nucleic acid, essential fatty acid, and other known functional food materials may be appropriately arbitrarily mixed.

The production method of food compositions in various forms is not particularly limited, and a known method can be used appropriately. Further, a component capable of reducing the peculiar taste and smell of a herbal medicine may be added, or treatment with such a component may be performed.

The food composition of the present embodiment may also be a food for the purpose of preservation of health, health maintenance, health promotion, and the like, for example, a health food, a food with function claims, a nutritional supplementary food, a supplement, a food for specified health uses, a food with nutrient function claims, or the like. In a case of being a supplement, the food composition of the present embodiment may be in a form of, for example, tablets, capsules, granules, a liquid, a powder, a drink, jelly, or the like. The food composition of the present embodiment may be ingested as it is, or may be mixed into meals, snacks, beverages, or the like.

The contents of components (1), (2), (3), and (4) as active components in the food composition of the present embodiment are not particularly limited, and vary depending on the form of the food composition. For example, in a case where the food composition is in a form of tablets/capsules/granules/a liquid/a powder/a supplement such as stick jelly, the total weight of each herbal medicine in the food composition can be mentioned to be typically more than 0 to 100% by weight, for example, 10% to 80% by mass of the food composition. Further, in a case where the form of the food composition is a form of various kinds of beverages/jelly, or a form of general foods such as foods/retort pouch foods, the total weight of each herbal medicine in the food composition can be mentioned to be typically 0.05 to 25% by mass of the food composition. Considering the taste and the amount, it is preferable to minimize the mixing amounts of the active components in a food composition, and in that case, it is preferable to use an extract of a herbal medicine rather than mixing the herbal medicine as it is. Since the herbal medicine contained in the food composition of the present embodiment has usually a mild effect when used as a food, the intake is not particularly limited. If the intake of the food composition of the present embodiment is an amount that includes the daily doses of the above-described herbal medicines, the desired effects of preventing, improving, or alleviating symptoms relating to qi deficiency and/or blood deficiency can be obtained.

The pharmaceutical composition of the present embodiment may be in a form of a drink, syrup, jelly, a lyophilized powder, tablets, capsules, pills, a powder, fine granules, granules, or the like, and may contain in addition to the components (1), (2), (3), and (4) as the active components, for example, a pharmaceutically acceptable component, for example, an excipient, a binder, a lubricant, a disintegrant, an emulsifier, a surfactant, a base, a dissolution aid, or a suspending agent.

The composition of the present embodiment may also be used in combination with other compositions. For example, by using the composition of the present embodiment in combination with an over-the-counter drug that is used to treat symptoms relating to qi deficiency and/or blood deficiency, for example, a vitamin B, a liver hydrolysate, or the like, discomfort symptoms can be eliminated with minimal use of the over-the-counter drug.

The composition may be ingested once a day, or may be ingested in several divided doses, for example, 2, 3 or 4 divided doses. In a case of ingesting the composition in several divided doses, the composition should be ingested so that the total doses are the daily dose. The timing of the ingestion of the composition of the present embodiment is not particularly limited, and in a case where the ingestion is once a day, the composition may be taken at any time such as after waking up, before meal, between meals, after meal, or before going to bed, and in a case where the ingestion is several times a day, the composition may be taken at two, three, or four or more of timings of after waking up, before meal, between meals, after meal, and before going to bed. Further, the composition may be ingested together with meal.

The composition of the present embodiment may be ingested continuously over a period of one or more weeks, two or more weeks, four or more weeks, three or more months, six or more months, or one or more years. Since the composition of the present embodiment is a safe herbal medicine component, it is safe to ingest the composition over a long period of time, and by taking the composition for a long period of time, the physical constitution is gradually improved, and the symptoms relating to qi deficiency and/or blood deficiency can be gradually alleviated or improved.

The method of one embodiment is a method for preventing, improving, or alleviating symptoms relating to qi deficiency and/or blood deficiency, including a step of administering the composition of the present embodiment to a subject in need thereof. In this regard, the subject is a subject who is aware of symptoms relating to qi deficiency and/or blood deficiency, or have been diagnosed with qi deficiency and/or blood deficiency based on Chinese medicine. The composition is given (for example, administered) to the subject in the daily dose described above. The administration period is not particularly limited, and may be one or more weeks, two or more weeks, four or more weeks, three or more months, six or more months, or one or more years.

The composition of one embodiment is a composition for use in preventing, improving, or alleviating symptoms relating to qi deficiency and/or blood deficiency, and the composition may be a composition of the above present embodiment. In this regard, the use method (intake, and intake period) is as described above.

The use of one embodiment is use of the composition of the present embodiment, in producing a composition for preventing, improving, or alleviating symptoms relating to qi deficiency and/or blood deficiency. Further, the use of another embodiment is use of the composition of the present embodiment, in preventing, improving, or alleviating symptoms relating to qi deficiency and/or blood deficiency. In this regard, the use method (intake, and intake period) is as described above. The composition may be a food composition or a pharmaceutical composition, and the composition can be produced according to the usual method so as to include an effective amount of herbal medicine or herbal medicine extract of the components (1), (2), (3), and (4), and may include other components described above if necessary.

Hereinafter, examples will be given in order to describe the present invention further in detail. However, the present invention should not be limited at all to these examples.

### EXAMPLES

### Test Example 1

(Evaluation Test of Effect on Physical Disorder Relating to Qi Deficiency and/or Blood Deficiency)

A single-blind controlled before-after trial was conducted on 19 individuals (Example 1), 18 individuals (Example 2 and Comparative Example 1), or 14 individuals (Example 3), who were Japanese men and women aged 18 or more to 64 or less and had been aware of daily physical disorders relating to qi deficiency and/or blood deficiency, and the effects of the present invention were investigated.

### Composition

The composition of herbal medicines and the amount used for one dose of Examples 1, 2, and 3, and Comparative Example 1 are shown in Table 1. As the raw material herbal medicines, herbal medicines listed in Table 2 were mixed according to the proportion of one dose of the herbal medicines, the mixture was boiled and extracted in water, the filtration, concentration, and sterilization were performed, dextrin in an adequate amount was added to the obtained product, and then the resultant product was dried with a spray dryer to obtain a mixed plant extract powder. In the obtained extracts, as the raw material herbal medicine, 6.0 g of extract powder of Example 1, 15.0 g of extract powder of Example 2, 7.2 g of extract powder of Example 3, or 6.0 g of extract powder of Comparative Example 1, equivalent to the amount of herbal medicines listed in Table 2 was used as a food composition for one day.

TABLE 1

| | Component | Component | Component | Component |
|---|---|---|---|---|
| | (1)/g | (2)/g | (3)/g | (4)/g |
| Example 1 | Honey-fried | Wild | Tea leaf/3 | Ripe tangerine peel/3 |
| | licorice root/3 | turmeric/3 | | Psoralea corylifolia |
| | | | | fruit/3 |
| | | | | Chaenomeles speciosa |
| | | | | fruit/3 |
| | | | | Piper longum/1.5 |
| | | | | Eleutherococcus |
| | | | | Senticosus Rhizome/3 |
| Example 2 | Malt starch | Zedoary | Chrysanthemum | processed ginger/3 |
| | syrup/10 | root/3 | flower/6 | Coriander/3 |
| | Dioscorea | | | |
| | japonica | | | |
| | Thunberg/9 | | | |
| Example 3 | Dioscorea | Safflower/3 | Mentha | Perilla/6 |
| | japonica | | herb/1.5 | Cinnamon/3 |
| | Thunberg/9 | | | Coix seed/9 |
| | Honey-fried | | | Corn silk/15 |
| | licorice root/3 | | | |
| Comparative | Malt starch | Zedoary | Chrysanthemum | processed ginger/3 |
| Example 1 | syrup/3 | root/3 | flower/6 | |

Each of subjects ingested the daily dose of extract powder of Example 1, 2, or 3 or Comparative Example 1 (Example 1: 2.0 g, Example 2: 5.0 g, Example 3: 2.4 g, and Comparative Example 1: 2.0 g) continuously for two weeks (14 days) once a day before breakfast after dissolving the powder in about 150 cc of warm water. Discomfort questionnaires were conducted for all the subjects before the ingestion and at the completion of the ingestion for 2 weeks, and the degree of improvement in the symptoms was evaluated. In the discomfort questionnaires before the ingestion, for each symptom group listed in Table 2, scores of 3 levels: “extremely worried (1)”, “slightly worried (2)”, and “not worried (3)” were set, and the presence or absence of awareness of each symptom was evaluated. In the discomfort questionnaires at the completion of the ingestion, for each symptom group listed in Table 2, scores of 5 levels: “very improved (1)”, “improved (2)”, “unchanged (3)”, “impaired (4)”, and “very impaired (5)” were set, and the presence or absence of improvement of each symptom was evaluated. The proportion (%) of the subjects of which each score of the questionnaire at the completion of the ingestion was 4 or 5 among the subjects of which each score of the questionnaire before the ingestion was 1 or 2 was taken as the improvement rate, and it was determined that the extract had an effect on the improvement of the discomfort symptom having an improvement rate of 10% or more, and the extract had a remarkable effect on the improvement of the discomfort symptom having an improvement rate of 30% or more.

### Results

The results are shown in Table 2. From Table 2, as compared with the subjects who ingested the herbal medicine extract of Comparative Example 1 that contains component (1) including one herbal medicine, component (2) including one herbal medicine, component (3) including one herbal medicine, and one kind of a herbal medicine constituting component (4), in the subjects who ingested the herbal medicine extract of Example 1 that contains component (1) including one herbal medicine, component (2) including one herbal medicine, component (3) including one herbal medicine, and component (4) including five herbal medicines, Example 2 that contains component (1) including two herbal medicines, component (2) including one herbal medicine, component (3) including one herbal medicine, and component (4) including two herbal medicines, or Example 3 that contains component (1) including two herbal medicines, component (2) including one herbal medicine, component (3) including one herbal medicine, and component (4) including four herbal medicines, improvements were observed in chronic fatigue, fatigability, malaise, listlessness, abnormality in urine volume, insomnia, and sleep disorder. In addition to the above effects, it was observed that weakness, anorexia, diarrhea, urinary incontinence, frequent urination, cold extremity, feeling of cold, susceptibility to infection, excessive sweating, and ill complexion were improved by the ingestion of the herbal medicine extract of Example 1; weakness, diarrhea, and frequent urination were improved by the ingestion of the herbal medicine extract of Example 2; and feeling of cold was also improved by the ingestion of the herbal medicine extract of Example 3. That is, the effects on the prevention, improvement, or alleviation were confirmed in the whole symptoms relating to qi deficiency and/or blood deficiency based on the synergistic action of the components (1), (2), (3), and (4).

TABLE 2

| | | | Easy to get | Heavy and | Numbness | | Lack of | | Low amount |
|---|---|---|---|---|---|---|---|---|---|
| | | | tired/shortness | sluggish in | of the body | | appetite/ | | of urine |
| | | Chronic | of breath | the body | and limbs/ | | heartburn | | (abnormality in |
| Group | Item | fatigue | (fatigability) | (malaise) | weakness | Listlessness | (anorexia) | Diarrhea | urine volume) |
| Example 1 | Improvement | 47 | 58 | 50 | 50 | 17 | 33 | 25 | 50 |
| | rate (%) | | | | | | | | |
| | Improvement | ⊙ | ⊙ | ⊙ | ⊙ | ◯ | ⊙ | ◯ | ⊙ |
| Example 2 | Improvement | 36 | 20 | 33 | 33 | 27 | 0 | 25 | 100 |
| | rate (%) | | | | | | | | |
| | Improvement | ⊙ | ◯ | ⊙ | ⊙ | ◯ | | ◯ | ⊙ |
| Example 3 | Improvement | 23 | 11 | 20 | 0 | 13 | 0 | 0 | 100 |
| | rate (%) | | | | | | | | |
| | Improvement | ◯ | ◯ | ◯ | | ◯ | | | ⊙ |
| Comparative | Improvement | 25 | 9 | 25 | 0 | 0 | 0 | 0 | 0 |
| Example 1 | rate (%) | | | | | | | | |
| | Improvement | ◯ | | ◯ | | | | | |

| | | Urine | High frequency | | Low body | | Easy to | Symptoms |
|---|---|---|---|---|---|---|---|---|
| | | leakage | of urination | Cold limbs | temperature | Easy | catch a cold | of a cold |
| | | (urinary | (frequent | (cold | (feeling | to get | (susceptibility | (chills/runny |
| Group | Item | incontinence) | urination) | extremity) | of cold) | sick | to infection) | nose/cough) |
| Example 1 | Improvement | 25 | 20 | 13 | 25 | 33 | 20 | 17 |
| | rate (%) | | | | | | | |
| | Improvement | ◯ | ◯ | ◯ | ◯ | ⊙ | ◯ | ◯ |
| Example 2 | Improvement | 0 | 50 | 0 | 0 | 0 | 0 | 0 |
| | rate (%) | | | | | | | |
| | Improvement | | ⊙ | | | | | |
| Example 3 | Improvement | 0 | 0 | 0 | 50 | 0 | 0 | 0 |
| | rate (%) | | | | | | | |
| | Improvement | | | | ⊙ | | | |
| Comparative | Improvement | 0 | 0 | 0 | 0 | 0 | 0 | 0 |
| Example 1 | rate (%) | | | | | | | |
| | Improvement | | | | | | | |

| | | Difficulty | Poor sleep/ | | | Pale |
|---|---|---|---|---|---|---|
| | | in falling | waking up | Lots of | | complexion |
| | | asleep | during the night | night | Excessive | (ill |
| Group | Item | (insomnia) | (sleep disorder) | sweats | sweating | complexion) |
| Example 1 | Improvement | 40 | 56 | 25 | 33 | 50 |
| | rate (%) | | | | | |
| | Improvement | ⊙ | ⊙ | ◯ | ⊙ | ⊙ |
| Example 2 | Improvement | 38 | 33 | 0 | 0 | 0 |
| | rate (%) | | | | | |
| | Improvement | ⊙ | ⊙ | | | |
| Example 3 | Improvement | 43 | 20 | 0 | 0 | 0 |
| | rate (%) | | | | | |
| | Improvement | ⊙ | ◯ | | | |
| Comparative | Improvement | 29 | 13 | 0 | 0 | 0 |
| Example 1 | rate (%) | | | | | |
| | Improvement | ◯ | ◯ | | | |

—: The questionnaire was not conducted for Example 1
⊙: There is a remarkable effect on the improvement
◯: There is an effect on the improvement

1. A composition, comprising as active components the following components (1), (2), (3), and (4):
component (1): at least one kind of herbal medicine selected from the group consisting of honey-fried licorice root, licorice root, Dioscorea japonica Thunberg, jujube fruit, malt starch syrup, and ginseng root;
component (2): at least one kind of herbal medicine selected from the group consisting of wild turmeric, longan aril, zedoary root, Suberect Spatholobus stem, Luffa vegetable sponge, Sparganium rhizome, safflower, and Artemisia anomala;
component (3): at least one kind of herbal medicine selected from the group consisting of tea leaf, mentha herb, mulberry leaf, chrysanthemum flower, and Koushi; and
component (4): at least two kinds of herbal medicines selected from the group consisting of ripe tangerine peel, Psoralea corylifolia fruit, Chaenomeles speciosa fruit, Piper longum, cinnamon twig, Perilla, ginger root, coriander, Allium fistulosum Linne, Zizyphi Spinosi Semen, Chinese arborvitae kernel, plantago seed, plantago herb, coix seed, corn silk, belvedere Fruit, Benincasa seed, knotgrass, snowbellleaf tickclover herb, cinnamon, processed ginger, fennel, Zanthoxylum fruit, Piper nigrum, Eleutherococcus Senticosus Rhizome, and Artemisia leaf.

2. The composition according to claim 1, wherein the composition is for use in preventing, improving, or alleviating symptoms relating to qi deficiency and/or blood deficiency.

3. The composition according to claim 2, wherein the symptoms relating to qi deficiency and/or blood deficiency is at least one selected from the group consisting of chronic fatigue, fatigability, malaise, listlessness, abnormality in urine volume, frequent urination, insomnia, and sleep disorder.

4. The composition according to claim 1, wherein the composition is a food composition or a pharmaceutical composition.
